(19) United States
(12) Patent Application Publication
(10) Pub. No.: US 2021/0295369 A1
(43) Pub. Date: Sep. 23, 2021

(54) SYSTEM AND METHOD FOR FACILITATING AND MANAGING PATIENT PAYMENTS AND DISCOUNTS RELATED TO HEALTHCARE MARKETPLACE TRANSACTIONS

(71) Applicant: Kalderos, Inc., Chicago, IL (US)

(72) Inventors: Jeremy Docken, Chicago, IL (US); Micah Litow, Chicag, IL (US)

(73) Assignee: Kalderos, Inc., Chicago, IL (US)

(21) Appl. No.: 17/019,204

(22) Filed: Sep. 12, 2020

Related U.S. Application Data
(63) Continuation-in-part of application No. 16/824,455, filed on Mar. 19, 2020.

(51) Int. Cl. G06Q 30/02; G16H 40/00; G16H 20/10; G06Q 40/08; G06Q 20/36

(52) U.S. Cl. CPC G06Q 30/0234; G16H 40/00; G16H 20/10; G06Q 30/0279; G06Q 40/08; G06Q 20/3678; G06Q 30/0215

(57) ABSTRACT

A system and method are disclosed for effectuating a payment to a patient relating to the cost of pharmaceutical product between a discount payer, having a computing system including a processor, a memory, and an internet connection, and a patient. The method includes the steps of providing a discount manager, the discount manager having a computing system including a processor, a memory, and an internet connection, the discount manager facilitating the relationship between the discount payer and the patient; the system includes allowing all parties to align on, discuss, and debate who should receive a discount; the discount payer providing a payment, based on stakeholder alignment, to the patient for acquisition of the pharmaceutical product; and the patient receiving the payment, the payment received in the form of cash to the patient or as a credit to the discount manager for future use by the patient.

### CROSS-REFERENCE TO RELATED APPLICATION

This application is a continuation-in-part of U.S. patent application Ser. No. 16/824,455, for a “System and Method for Facilitating and Managing Secondary Transactions Related to Healthcare Marketplaces,” filed Mar. 19, 2020, which claims the benefit of priority to U.S. Provisional Patent Application No. 62/820,945 for a “System and Method for Facilitating and Managing Discounts Relating to the Distribution and Sales of Pharmaceuticals,” filed Mar. 20, 2019, all of which are incorporated by reference in their entireties herein.

### TECHNICAL FIELD

The present invention relates to a system and method for facilitating and managing patient payments and discounts related to healthcare marketplace transactions. More specific, the present invention relates to a novel model for facilitating payments and/or discounts directly between a drug manufacturer (“Discount Payer”) and a patient relating to pharmaceuticals, medical devices, medical diagnostics, medical procedures and the like, which may be paid directly or indirectly through a novel entity, a “Discount Manager,” in an expeditious manner.

### BACKGROUND

The domestic pharmaceutical supply chain provides for certain systems and methods for distributing pharmaceutical products from a drug manufacturer to a pharmacy or physician office, which are ultimately dispensed to a consumer or patient, and for obtaining payment from the patient and a patient's health insurance plan back through the supply chain to the pharmacy or physician office, drug manufacturer, and various other intermediaries.

Typically in the conventional pharmaceutical supply chain, pharmaceutical products are sold by the drug manufacturer to a wholesaler, which then fulfills replenishment orders at pharmacies or physician offices. As prescriptions are filled, a pharmacy benefits manager working with the pharmacy and a particular patient's health insurance plan attempts to coordinate pricing and reimbursement rates to be sure the pharmacy is ultimately fairly compensated for filling the prescription, the health insurance plan receives discounts or rebates from the drug manufacturer, and the patient pays the appropriate out-of-pocket cost.

The conventional systems and methods involved in the domestic pharmaceutical supply chain for effectuating discounts focus on one or a select handful of preferred pharmaceutical discount programs and do not take into account the entire discount program universe, including the coordination of discounts stemming from or mandated by numerous programs, including but not limited to, federal programs such as the Medicare Prescription Drug Benefit (often referred to as “Medicare Part D” or simply “Part D” coverage), state programs such as the Medicaid Drug Rebate Program (“Medicaid”), commercial managed care formulary discounts, and/or the so-called “340B” drug discounts stemming from Section 340B of the Public Health Service Act of 1992.

None of the conventional systems and methods involved in the domestic pharmaceutical supply chain provide for a system-level solution that provides for the effective coordination of all drug discount programs and/or provides for a point-of-sale discount between the drug manufacturer and the pharmacy, physician's office, and/or patient. Consequently, double-discounting and overpayment/underpayment for drugs is a typical inefficiency in the conventional domestic pharmaceutical supply chain.

Moreover, drug manufacturers (“Discount Payers”) seeking to deliver discounts directly to patients through the form of a so-called “co-pay coupon” often are blocked by Pharmaceutical Benefits Managers (“PBM”), which have increasingly started using co-pay accumulators to deny patients the ability to use co-pay coupon benefits towards satisfying the patient's deductible.

A Jun. 19, 2020 proposed rule for the Centers for Medicare & Medicaid Services, Department of Health and Human Services (“HHS”), 42 CFR Parts 433, 438, 447, and 456, if approved, would fundamentally change how drug manufacturers design and operationalize patient discounts, such as co-pay coupon programs, by requiring that patients receive the entire benefit of manufacturer-provided co-pay support.

As a result of the ineffectiveness of conventional systems and methods of patient discounts and co-pay coupon programs, there presently is no way for a drug manufacturer or other entity to directly fund, or provide discounts to, a patient for the provision of pharmaceuticals, medical devices, medical diagnostics, or medical procedures. There presently is no system or method in the marketplace that is immediately compliant with the HHS proposed rule.

### SUMMARY OF THE INVENTION

According to one non-limiting aspect of the present disclosure, an example embodiment of a method is disclosed for effectuating a payment to a patient relating to the cost of pharmaceutical product between a discount payer or discount stakeholder, having a computing system including a processor, a memory, and an internet connection, and a patient. The method includes the steps of providing a discount manager, the discount manager having a computing system including a processor, a memory, and an internet connection, the discount manager facilitating the relationship between the discount payer and the patient; the discount payer providing a payment to the patient for acquisition of the pharmaceutical product; and the patient receiving the payment, the payment received in the form of cash to the patient or as a credit to the discount manager for future use by the patient.

According to another non-limiting aspect of the present disclosure, an example embodiment of a system is disclosed for effectuating a payment to a patient relating to the cost of pharmaceutical product between a discount payer, having a computing system including a processor, a memory, and an internet connection, and a patient. The system includes a discount manager for facilitating payment between the discount payer and the patient, the discount manager having a computing system including a processor, a memory, and an interne connection, the discount manager having a relationship with the discount payer and a relationship with the patient; a patient-facing app provided by the discount manager, the app downloadable to and executable by a smart phone device accessible to the patient, the app being interconnected with a cloud-based network having a database storage for storing an account relating to the patient and for storing any healthcare insurance information relating to the patient; a rules matrix maintained by the discount manager and interconnected with the app, the rules matrix containing benefit information relating to any healthcare insurance of the patient; a virtual wallet residing within the app, the virtual wallet being configured to receive and hold payment or credit relating to said pharmaceutical product from the discount payer and accessible to the patient; wherein the discount manager facilitates payment from the discount payer to the patient via the virtual wallet based on applicable benefit rules contained within the rules matrix.

Additional features and advantages are described herein, and will be apparent from the following Detailed Description and the figures.

### BRIEF DESCRIPTION OF THE DRAWINGS

Features and advantages of the system and method for facilitating and managing discounts in the pharmaceutical supply chain described herein may be better understood by reference to the accompanying drawings in which:

FIG. 1 depicts the conventional flow of products and financial transactions among parties in the domestic commercial pharmaceutical supply chain;

FIG. 2 depicts the flow of products and financial transactions between a discount payer and a discount recipient;

FIG. 3 depicts the flow of products and financial transactions between the discount recipient, a discount stakeholder, and a patient;

FIG. 4 depicts a novel discount chargeback model and the exchange of information and financial transactions between the discount payer and the discount recipient;

FIG. 5 depicts the agreement framework between the discount payer, discount manager, and discount recipient that provides for the novel discount chargeback model of FIG. 4;

FIG. 6 depicts an advantage of the novel discount chargeback model of FIG. 4, in which the model intercepts and prevents impermissible double discounting that is typical in the conventional pharmaceutical supply chain of FIG. 1;

FIG. 7 depicts the architecture concept of an electronic environment of a discount manager facilitating the novel patient discount method disclosed herein; and

FIG. 8 depicts the interaction of an electronic environment of a discount manager facilitating the novel patient discount method disclosed herein.

A skilled artisan will appreciate the foregoing details, as well as others, upon considering the following Detailed Description of certain non-limiting embodiments of the system and method for facilitating and managing patient payments and/or discounts according to the present disclosure. One of ordinary skill also may comprehend certain of such additional details upon using the systems and methods described herein.

### DETAILED DESCRIPTION

The present disclosure, in part, is directed to a system and method for facilitating and managing patient payments and discounts related to healthcare marketplace transactions. More specific, the present invention relates to a novel model for facilitating payments and/or discounts directly between a drug manufacturer (“discount payer”), or through a discount recipient or other covered entity, and a patient relating to pharmaceuticals, medical devices, medical diagnostics, medical procedures and the like, which may be paid directly or indirectly through a novel entity, a “discount manager,” in an expeditious manner.

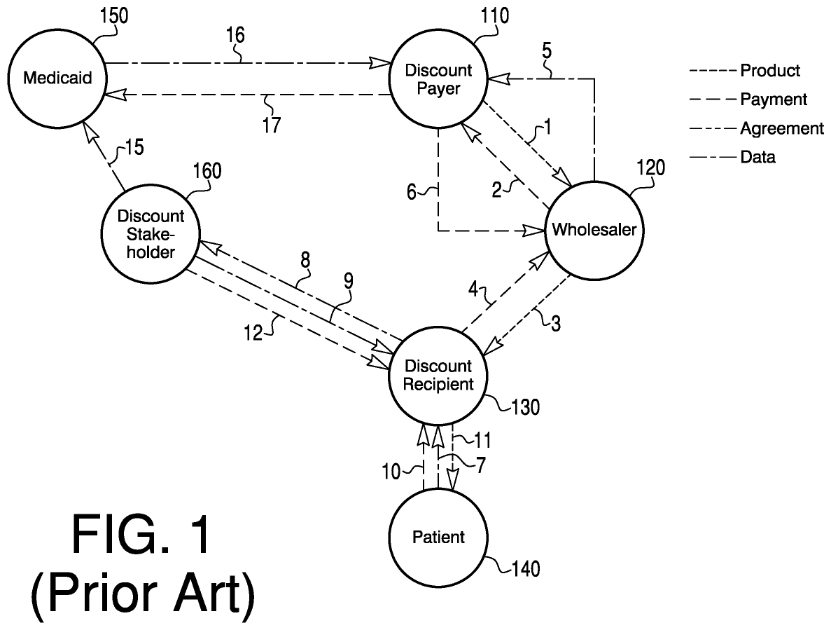

As shown in FIG. 1, a conventional flow of products and financial transactions among parties in the domestic commercial pharmaceutical supply chain is shown. With reference to the numerals shown on FIG. 1, a drug manufacturer or discount payer 110 ships ordered pharmaceutical products, as indicated by reference numeral 1, to a pharmaceutical wholesaler 120. In turn, the wholesaler 120 pays to the discount payer 110 an undiscounted list price, as indicated by reference numeral 2. The wholesaler 120 ships the pharmaceutical product to a pharmacy or discount recipient 130, as shown by reference numeral 3, upon receiving a replenishment order from the discount recipient 130. The discount recipient 130 pays the wholesaler 120 for the product at the price mandated by Section 340B (the “340B price”), as shown by reference numeral 4, and then the wholesaler 120 passes a chargeback request to the discount payer 110 based on the 340B price, as shown by reference numeral 5. As shown by reference numeral 6, the discount payer 110 pays the wholesaler 120 the difference between the undiscounted list price and the 340B price paid by the discount recipient 130 for the pharmaceutical products. One skilled in the art would appreciate that the transaction between the discount payer 110 and the wholesaler 120, as one example, may be made via direct payment or through reconciliation of credit memos.

Typically, at this point, with the pharmaceutical product at the pharmacy or discount recipient 130, the patient 140 provides the discount recipient 130 with the patient's insurance information, as shown by reference numeral 7. The discount recipient 130 will check if the patient 140 is eligible to receive the pharmaceutical product with the discount stakeholder 160, which may comprise a pharmacy benefits manager (“PBM”), as well as communicate the discount recipient's 130 acquisition cost for the pharmaceutical product, as shown by reference numeral 8. The discount stakeholder 160 will send a return message back to the discount recipient 130 authorizing the discount recipient 130 to dispense the product to the consumer or patient 140, the copayment or coinsurance that the discount recipient 130 must collect from the consumer or patient 140, as well as the payment amount the discount stakeholder 160 will reimburse the discount recipient 130 (which is usual and customary to base off of the discount recipient 130 communicated acquisition cost), as shown by reference numeral 9. After collecting the copayment or coinsurance from the consumer or patient 140, as shown by reference numeral 10, the discount recipient 130 dispenses the pharmaceutical product to the consumer or patient, as shown by reference number 11. The discount stakeholder 160 will pay the discount receipt 130 the communicated reimbursement amount, as shown by reference number 12.

With conventional systems, the eligibility of the 340B price is determined post-adjudication, meaning that discounts are applied when product is dispensed to the patient based on assumptions made by the discount recipient and the wholesaler that may be incorrect, leading to duplicate or erroneous discounts paid out by the discount payer 110. For instance, data regarding a pharmaceutical transaction is processed after the product is dispensed to a consumer or patient to evaluate if the initial undiscounted list price transaction met the 340B price eligibility criteria. If eligibility is determined to be correct, then the discount recipient 130 will initiate a “340B replenishment order” with the wholesaler 120 at the 340B price, leading the discount payer 110 to pay a 340B chargeback to the wholesaler 120. The discount recipient 130 is usually required to update the acquisition cost originally communicated, as shown by reference number 8, to indicate that the acquisition cost was a discounted 340B price, but this process is prone to error, or simply does not occur. As a result, the discount stakeholder 160 often requests a commercial discount from the manufacturer, as shown by reference number 13, not knowing that the transaction had been filled with a product sold subject to a 340B discount (knowledge of which should cause the discount stakeholder 160 to not request a commercial discount). Consequently, the discount payer 110 frequently pays an erroneous duplicate discount, as shown by reference number 14.

In this particular scenario, as will become apparent in the conventional reimbursement process described herein, if the patient was covered by a state Medicaid plan 150, the discount stakeholder 160 will submit a summary of reimbursement transactions made during the quarter, as shown by reference numeral 15. Included in this summary is information regarding which reimbursements were made based on an undiscounted list price acquisition cost, and which reimbursements were made based on a 340B priced acquisition cost. Because the original acquisition cost communicated by the discount recipient 130 to the discount stakeholder 160, shown by reference numeral 8, is frequently incorrect, the state Medicaid program office 150 frequently submits an erroneous request for a Medicaid discount to the discount payer 110, as shown by reference numeral 16. In the conventional pharmaceutical supply chain, there is no effective mechanism in place for the discount payer 110 to know that the Medicaid discount requested by the state Medicaid program office 150 is requested on a transaction which the discount payer 110 already paid a 340B discount to the discount recipient 130. Consequently, the discount payer 110 frequently pays an erroneous duplicative discount to the state Medicaid program office 150, as shown by reference numeral 17.

The novel price concession model described herein involves the inclusion of a discount manager 170 to oversee the entire discounting universe. The inclusion of a discount manager 170 provides for many advantages described herein and which will become apparent to one of ordinary skill based on the following description in view of the accompanying drawings.

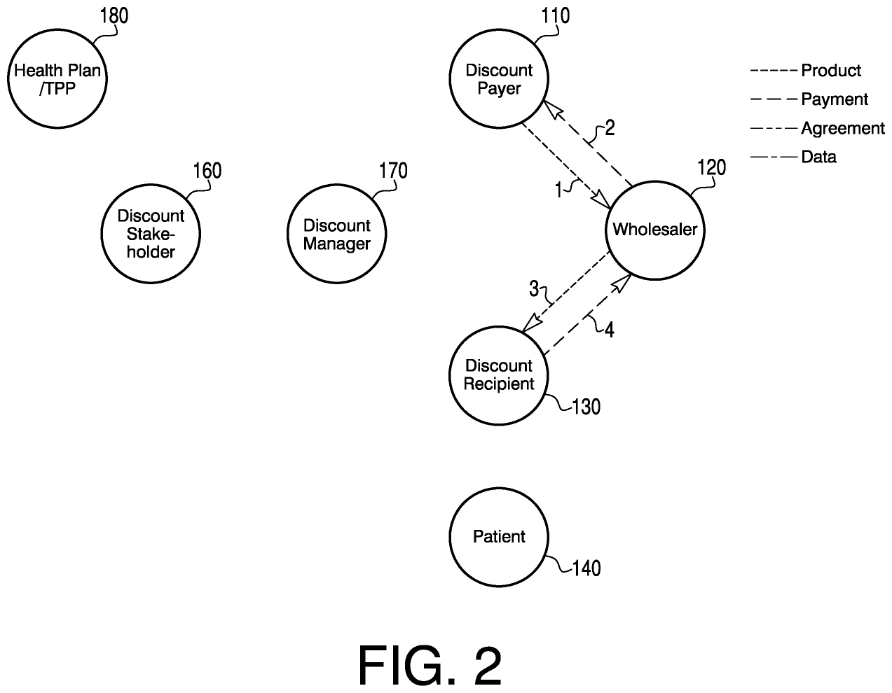

As shown in FIG. 2, the discount recipient 130 acquires pharmaceutical product from the discount payer 110 in much the same way as currently exists in the marketplace, whereby a wholesaler 120 purchases product from the discount payer 110 at an undiscounted list price. One important distinction in the acquisition process, however, is that the discount recipient 130 also purchases pharmaceutical product from the wholesaler 120 at the undiscounted list price, as shown by reference numeral 3, thereby eliminating the need for the wholesaler 120 to seek a chargeback request from the discount payer 110. This sets up an important advantage by the novel chargeback model described herein, in which the discount recipient 130 may directly request a chargeback from the discount payer 110 at the point-of-sale.

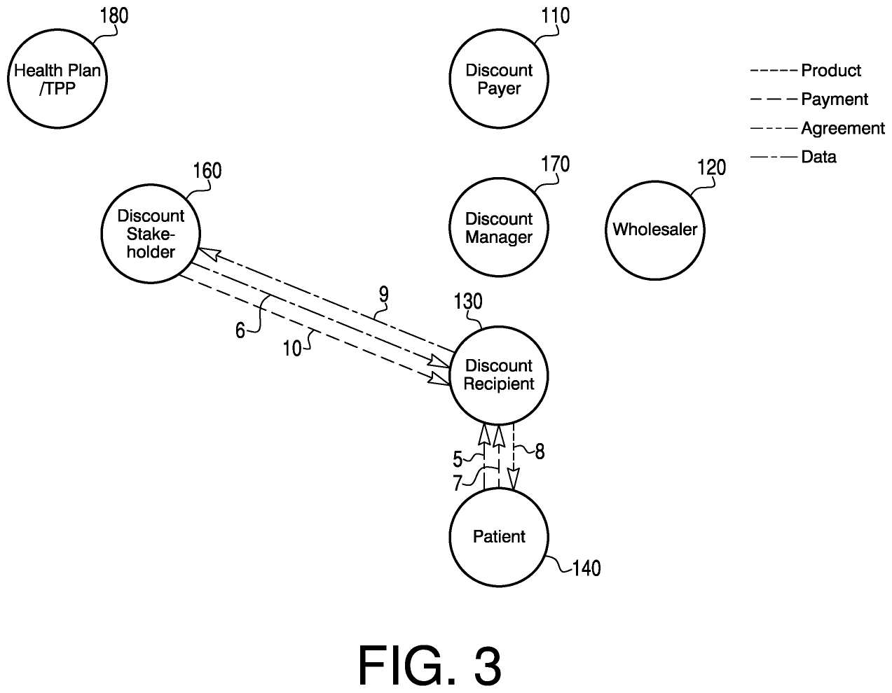

As shown in FIG. 3, the novel chargeback model described herein provides efficiencies in the dispensing of pharmaceutical product to the patient 140. The process begins at reference numeral 5 with the patient 140 presenting his or her insurance card to the discount recipient 130. The discount recipient 130 runs the insurance card through a switch interconnected with the discount stakeholder 160 at reference numeral 9, which approves the transaction at reference numeral 6 and returns to the discount recipient 130 the patient's fixed contract price and co-pay or co-insurance amount. The patient 140 pays the co-pay or co-insurance at reference numeral 7, and the discount recipient 130 provides the pharmaceutical product to the patient at reference numeral 8. The discount stakeholder 160, in turn, reimburses the discount recipient 130 the acquisition price of the pharmaceutical product at reference numeral 10.

The patient's 140 healthcare plan 180 may be a state Medicaid program 150, or may be any number of public, private, or governmental-sponsored programs, and the novel chargeback model described herein is configured to work with any discounting program, including but not limited to: 340B Drug Discount Programs, Veterans Affairs Pricing/Federal Supply Schedule, Wholesaler Sourcing, GPOs, Medicaid Drug Rebate Program, Commercial Managed Care Rebates, Medicare Part D Rebates, Medicare Coverage Gap, co-pay coupon cards, Patient Assistance Programs, Other Government Discounts (e.g., TRICARE, State Pharmaceutical Assistance Programs), and any other healthcare or discount programs that may come into existence in the future.

The interplay between the discount recipient 130 and the discount stakeholder 160 varies from one patient 140 to another, and is dependent, in part, upon the patient's healthcare plan 180 and the extent to which the patient may be enrolled in a government program, such as Medicaid or Medicare, and/or a private healthcare plan as primary healthcare or as a supplement to a government plan. In some instances, the discount recipient 130 may be the discount stakeholder 160, which submits a discount request to the discount payer 110. In instances where the discount recipient 130 is the PBM, the discounts often take the form of a rebate, which may or may not involve the transaction of payment but rather may be in the form of remittance, credit note, or some other change in the ledger between the parties. In other instances, the discount recipient 130 may be the consumer or patient 140, who may receive discounts from discount payer 110, discount stakeholder 160, health plan/TPP 180, and/or discount manager 170, which may be used towards healthcare services. In instances where the discount recipient 130 is the consumer or patient 140, the discounts may take the form of a deposit to a prepaid card or a health savings account. The complexity and number of relationships that may exist between the discount recipient 130 and the discount payer 110, including the presence of any discount stakeholder 160 separate from the discount recipient, are reasons why the state of the discounting universe is so uncertain and inefficient, and these reasons give rise to the need in the art for the novel chargeback model disclosed herein and facilitated by the discount manager 170 described in the present invention.

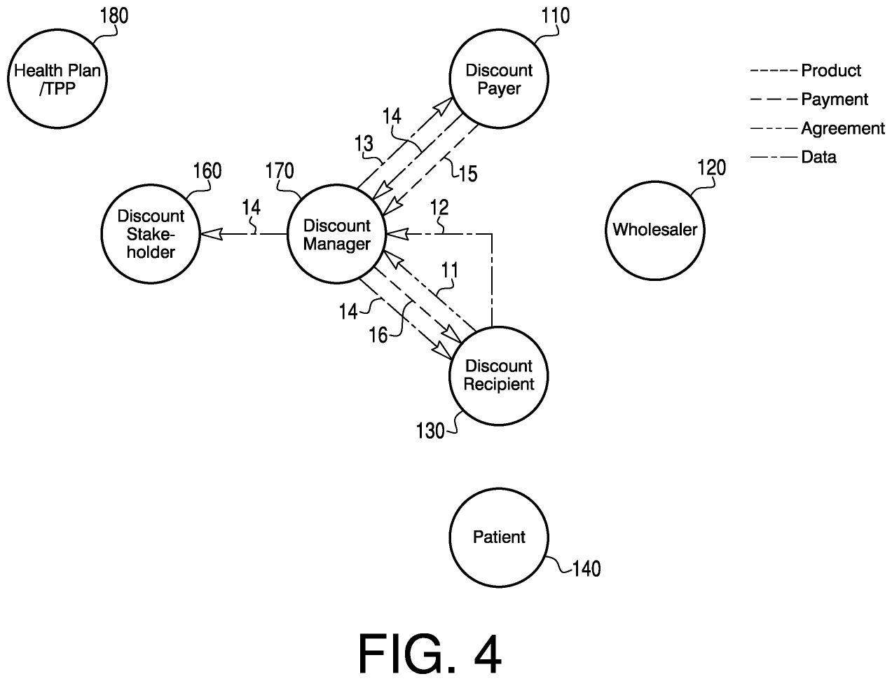

As shown in FIG. 4, the essence of the novel chargeback model described herein is facilitated by the discount manager 170. Participating discount recipients 130 enter into an agreement with the discount manager 170, as shown by reference numeral 11, similar to the agreements that a discount recipient 130 may enter into with a PBM or discount stakeholder 160.

The chargeback process is initiated when the discount recipient 130 submits a request for chargeback to the discount manager 170, as shown by reference numeral 12. The chargeback is calculated as the difference between the undiscounted list price and a discounted price. The discount manager 170 validates the chargeback request and provides a recommendation to the discount payer 110, as shown by reference numeral 13, to pay or fail the discount request in accordance with predefined rules set by the discount payer 110. The predefined rules may be self-imposed on the discount payer 110 or may result from applicable laws or contractual agreement entered into by the discount payer 110. The discount payer 110 resolves the discount request and notifies the discount manager 170, which relays the decision to the discount stakeholder 160 and the discount recipient 130, as shown by reference numeral 14. To the extent that the discount request is approved, the discount manager 170 facilitates the transfer of payment from the discount payer 110 to the discount recipient 130, as shown by reference numeral 16. In this arrangement, chargebacks flow directly from the drug manufacturer or discount payer 110 to the discount recipient 130.

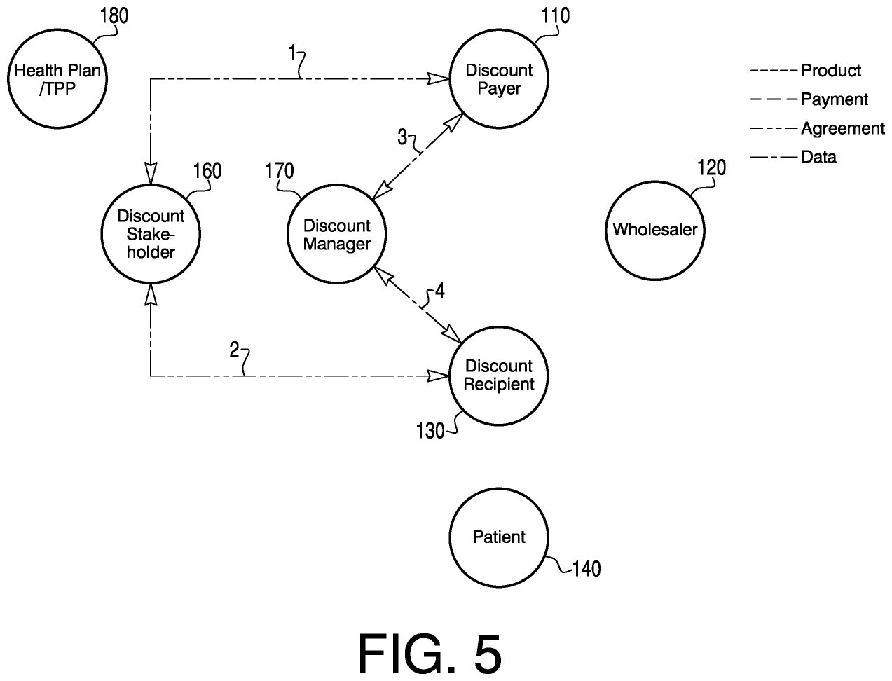

As shown in FIG. 5, an agreement framework is necessary to facilitate the novel chargeback model described herein, and to be sure that a dispensed product subject to a 340B price discount is not duplicatively discounted. As shown by reference numeral 1 in FIG. 5, the discount payer's 110 contract with the discount stakeholder 160 excludes contract pricing if the dispensed drug is subject to a 340B price discount. As shown by reference numeral 2, the discount stakeholder's 160 contract with the discount recipient 130 honors the discount payer's 110 contract exclusions relating to 340B price discounts. As shown by reference numeral 3, the discount payer's 110 contract with the discount manager 170 requires that the discount manager abide by the discount payer's 110 decision regarding contract and discount is applicable to a discount-eligible transaction. As shown by reference numeral 4, the discount manager's 170 agreement with the discount recipient 130 provides that a previously-paid discount may be reversed prior to new contract pricing being applied to the transaction if the discount recipient 130 attempts to re-price a previously-processed transaction.

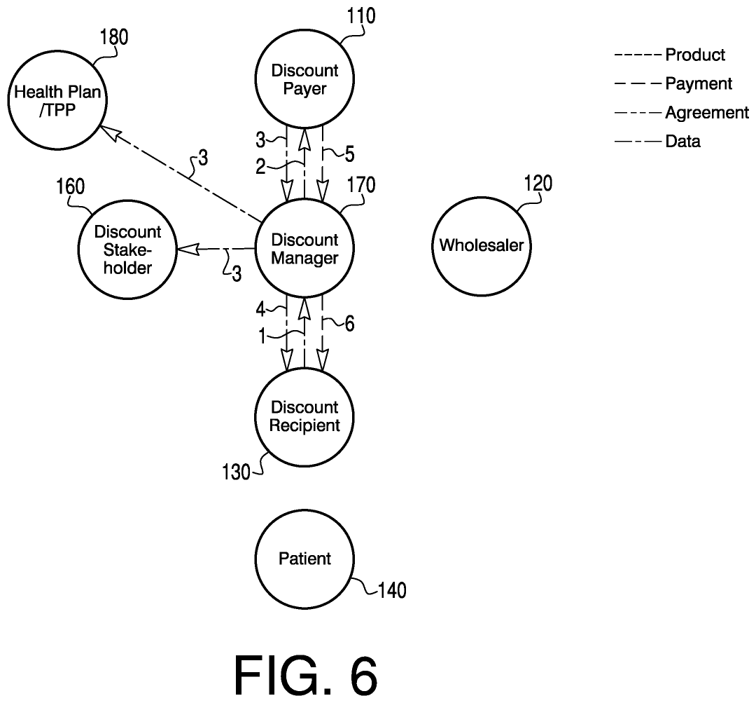

An advantage of the novel price concession model described herein is the proper policing and oversight of 340B price discounts required by federal law. As shown in FIG. 6, a discount recipient 130 or other covered entity may retroactively classify a script as being eligible for 340B pricing, at which point a 340B replenishment chargeback request is made by the discount recipient 130 to the discount manager 170, as shown by reference numeral 1. The discount manager 170 validates the request to determine if duplicate discounting (both 340B and PBM discounting) were applied, and if so, discount manager 170 notifies the discount payer 110, as shown by reference numeral 2. Because the 340B price discount is mandated by law, the discount payer 110 instructs the discount manager 170 to reverse or dispute any previously-paid discounts that have been requested by or paid to the discount stakeholder 160 and/or the health plan/TPP 180, as shown by reference numeral 3. The discount manager 170 notifies the discount recipient 130, as shown by reference numeral 4, that prior discounts paid to the discount stakeholder 160 and/or the health plan/TPP 180 have been reversed or disputed, and that the discount recipient 130 will be paid the requested 340B replenishment chargeback. The discount manager 170 facilitates payment of the net discount difference between the undiscounted list price and the 340B price, and payment is made from the discount payer 110 to the discount recipient 130, as shown by reference numeral 5.

Another advantage of the implementation of the discount manager 170 is the simplification of the payment process. The exchange of funds between the patient 140 and the manufacturer 110 occurs without passing through other stakeholders (such as, for example, a PBM), to ensure that funds are disbursed and used exclusively for the direct support of prescribing patients who are experiencing financial considerations that are restricting their access to a desired therapeutic (or, for example, medical test, procedure, or other treatment) prescribed or recommended by a physician or other medical professional.

Currently, manufacturers 110 attempt to provide discounts that directly benefit patients using co-pay coupons. A recent HHS proposed rule, “Medicaid Program: Establishing Minimum Standards in Medicaid State Drug Utilization Review and Supporting Value-Based Purchasing for Drugs Covered in Medicaid, Revising Medicaid Drug Rebate and Third Party Liability Requirements,” should it be finalized in current form, would require the manufacturer to guarantee, through the design of its co-pay coupon program, that the entire benefit of the manufacturer-provided co-pay support be enjoyed by the patient. Because co-pay coupon programs that are effectuated via the pharmacy switch are unable to be controlled by the manufacturer (i.e., the manufacturer is unable to mandate that the PBM be excluded from receiving data about the co-pay transaction), the proposed HHS rule would motivate manufacturers to seek a new model to manage co-pay coupon programs.

The discount manager 170 provides for a novel discount method that allows manufacturers 110 to design and effectuate direct cash payments to support patients following the purchase of a product in the event that a patient qualifies for financial support (per current standards).

The facilitation of discounting or direct payment between the manufacturer 110 and the patient 140 is carried out by means of a virtual wallet, which bypasses the conventional reimbursement process. The virtual wallet may be accessed by a patient 140 using any conventional electronic device, such as a desktop or laptop computer, tablet, or smart phone. In a preferred embodiment, the virtual wallet is a feature of an application (“app”) that may be downloaded from app stores such as the Apple® App Store® or the Google® Play® store and installed on and executed by a smart phone or tablet device.

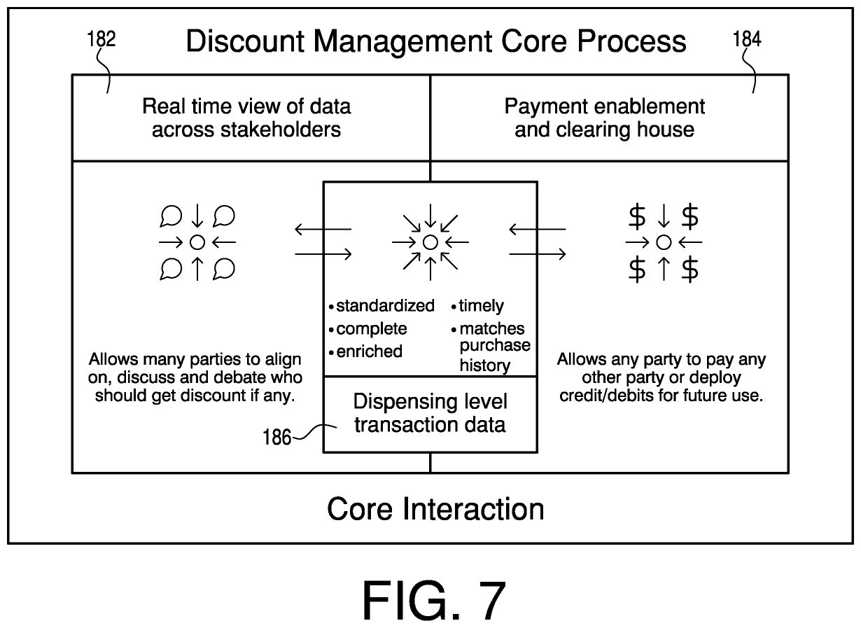

As shown in FIG. 7, an example of how the patient discount model works is disclosed. Specifically, the discount manager 170 is interconnected with and oversees two spheres of the patient discounting universe. On one hand, the discount manager 170 accesses a real time view of data across all stakeholders, as indicated at reference numeral 182. This allows a plurality of stakeholders and other parties to align on, discuss, and debate who should receive which discount, if any. On the other hand, the discount manager 170 maintains payment enablement, either acting as a payment provider itself or through partnership with a third-party payment partner, as shown at reference numeral 184. This allows any payer or other party to pay for the specific drug, diagnostic, or procedure of a patient, or to deploy credits or debits for future use. At the confluence of these two spheres, as shown at reference numeral 186, the discount manager 170 is best suited to effectuate patient payment and to timely dispense level transaction data that is standardized and complete.

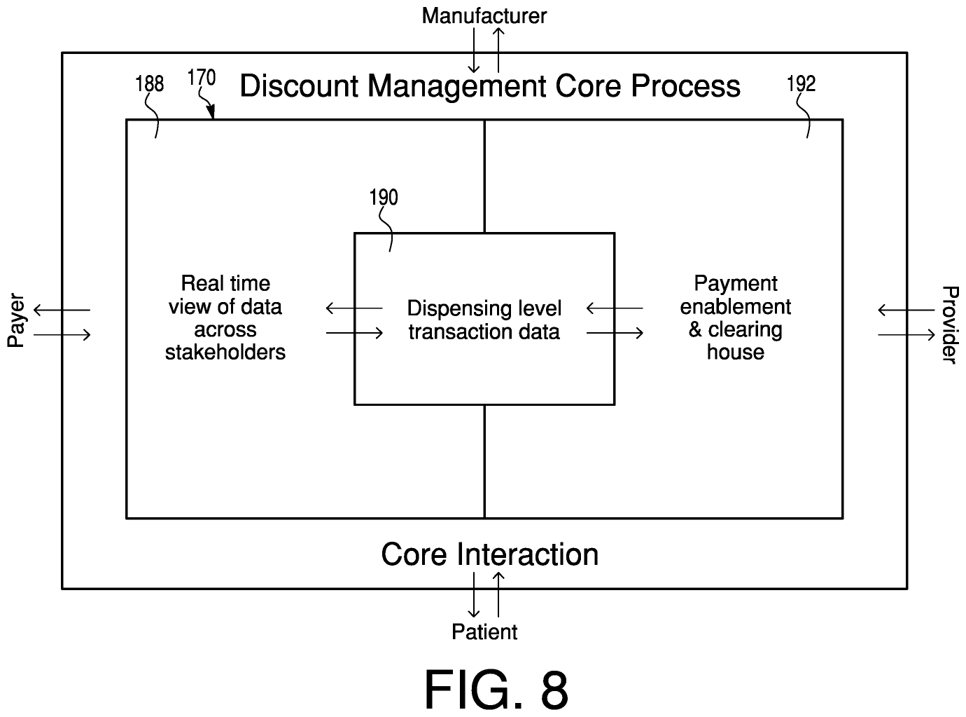

As shown in FIG. 8, an example of how the patient discount model effectuates payment is disclosed. The discount manager 170 lies at the center of the manufacturer 110, the provider 160, the payer, and the patient 140. In this arrangement, the discount manager 170 is able to take in all of the real time data of any stakeholder (as shown at reference numeral 188) and balance it with the needs and program eligibility of the patient to arrive at the best payment of discounting option for the patient, thereby delivering funds to the patient while at the same time delivering level transaction data (as shown at reference numeral 190) to the manufacturer 110 for improvement and refinement of the discounting process.

In effectuating payment or discounting through the virtual wallet (as shown at reference numeral 192), a patient 110 provides his or her medical healthcare insurance information to the facilitator of the virtual wallet, which may be the discount manager 170, which allows the discount manager 170 to identify if the patient is a governmental beneficiary (e.g., Medicaid, Medicare, TRICARE), commercial managed care beneficiary, or uninsured. If the patient 110 is uninsured, the patient provides the discount manager 170 proof of income required by manufacturers' patient assistance programs (“PAP”) so that the discount manager may register the patient with the PAP versus the co-pay coupon program.

A patient desiring to seek reimbursement or funds from the virtual wallet after filling a script with a pharmacy does so by photographing and uploading the prescription label and proof of purchase (i.e., receipt) via the virtual wallet app. The discount manager 170 evaluates what programs may be applicable for the patient (PAP, co-pay program, covered entity Rule 340B savings, and/or a non-profit organization or other payor). After verifying the program that provides the greatest benefit to the patient, the discount manager deposits funds into the patient's virtual wallet.

The discount manager 170 carries out these operations by serving as a central repository and interface for many other aspects of the pharmaceutical supply chain, including accounting, enterprise resource planning (“ERP”), contract management, business intelligence (“BI”), and other functions and applications. This delivery of information can be coordinated and carried out by offering ERP and BI solutions certain access via application programming interface (“API”) endpoints, electronic data interchange (“EDI”) connections, or other types of integration with the discount manager's database. For example, the discount manager 170 may send or receive information to other parties in the pharmaceutical supply chain using an API or EDI, external API, or through a flat file, spreadsheet, or other file or data type. Information managed by the discount manager 170 may be coded with identifiers from the National Council for Prescription Drug Programs (“NCPDP”) with respect to discount payers 110, discount recipients 130, and/or scripts. The discount manager 170 may serve as a data lake from which BI and ERP applications access, gather, and compile information.

The database or central repository of discount manager 170 is carried out on one or more servers, which may be cloud-based, interconnected with one another and with the world wide web and/or other network connections. An exemplary computing environment upon which embodiments of the present invention may be implemented includes a general-purpose computing system environment, such as computing system. In its most basic configuration, a computing system typically includes at least one processing unit and memory, and an address/data bus (or other interface) for communicating information. The computing system environment may be part of a cloud-based computing environment. For example, a storage host, a web server and an application engine may individually or collectively be implemented as or hosted on one or more such computing system environments. The memory is interconnected with at least one processor and includes instructions stored thereon which, when executed by the at least one processor, causes the processor to perform the operations of the discount manager 170, as described in the following set of paragraphs.

To carry out the functions of the virtual wallet, discount manager 170 integrates with pharmacies and other covered entities at the switch by passing a patient's BIN (“bank identification number”) and PCN (“primary care network”) numbers, just as if the patient passed an insurance card at the point of sale at a pharmacy. The BIN and PCN numbers are encoded on a physical electromagnetic swipe or RFID chip card encoded on a card presented by the patient at the point of sale and relating to, and uniquely identifying, the patient's virtual wallet. Alternatively, the BIN and PCN numbers may be passed at the point of sale by an electronic device, such as a smart phone, via Bluetooth® or other near-field communication to an electronic card reader or other point of sale device.

Data relating to a patient's virtual wallet account is maintained on the database or central repository of discount manager 170. The patient account may be accessible to a pharmacy or covered entity at the point of sale, or to a manufacturer, via an API extension from discount manager's databases.

The virtual wallet may be managed by a payment solution provider or third party separate from the discount manager 170, such that funds are disbursed in accordance with instructions from the discount manager but do not physically pass through the discount manager. Alternatively, it may be desirable or even necessary for the discount manager to manage the funding process such that funds do flow through the discount manager.

The script data obtained from the patient may be used by the discount manager 170 to identify discount stacking situations, to provide the manufacturer 110 with clarity into what additional discounts in addition to patient discounts may be applicable to the dispensing transaction.

Payers may be the manufacturer or discount payer 110, as described above, but also may be any number of other payers, including: non-profit organizations who deposit money into the virtual wallet to pay for specific drugs, treatments, or procedures; providers who want to give Rule 340B dollars, or split the Rule 340B payment with the patient; providers who share coupons; or any private citizen or organization that desires to pay for the cost of a specific drug, treatment, or procedure to a particular patient, such as, as an example, HEALfundr (healfundr.org).

While the models described herein have been described in detail with respect to the distribution, sales, discounting and payment of pharmaceutical products, the models disclosed herein are uniquely suited to work with other transactions related to healthcare marketplaces. For example, the models are entirely applicable with the distribution and sales of medical devices. Examples of medical devices include, but are not limited to: hospital beds, lifts, commode chairs; infusion pumps and supplies; blood glucose monitors and test strips; canes, crutches, walkers, and wheelchairs; oxygen, nebulizers, and nebulizer supplies and medications; continuous positive airway pressure machines and supplies; implantable medical devices such as pacemakers and defibrillators; and any other medical devices covered under healthcare or discount programs either defined by law or contract terms such as, but not limited to, value-based contracting arrangements. As another example, the models are entirely applicable to medical procedures, testing, diagnostics, and the like.

The patient discount model described herein provides for numerous advantages, some non-limiting examples of which are detailed as follows.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended set of claims.

What is claimed is:

1. A method for effectuating a payment to a patient relating to the cost of pharmaceutical product between a discount payer, having a computing system including a processor, a memory, and an internet connection, and a patient, the method comprising the steps of:
providing a discount manager, said discount manager having a computing system including a processor, a memory, and an internet connection, said discount manager facilitating said relationship between said discount payer and said patient;
said discount payer providing a payment to said patient for acquisition of the pharmaceutical product; and
said patient receiving said payment, said payment received in the form of cash to the patient or as a credit to the discount manager for future use by the patient.

2. The method of claim 1 wherein said discount payer is a covered entity under Rule 304B.

3. The method of claim 2 wherein said discount payer provides a portion of a discount received under Rule 304B to the patient.

4. The method of claim 1 wherein said discount payer is a charitable organization desiring to provide payment to said patient.

5. The method of claim 1 wherein said discount payer is a private fundraiser desiring to provide payment to said patient.

6. A method for determining eligibility of a patient for receiving a payment from a discount payer relating to the cost of a pharmaceutical product, the discount payer having a computing system including a processor, a memory, and an internet connection, the method comprising the steps of:
providing a discount manager, said discount manager having a computing system including a processor, a memory, and an internet connection, said discount manager facilitating said relationship between said discount payer and said patient;
said discount manager identifying whether the patient has any healthcare insurance;
said discount manager evaluating whether the patient is eligible for reimbursement or credit relating to the pharmaceutical product under any pharmaceutical reimbursement or discount program;
said discount manager selecting said pharmaceutical reimbursement or discount program that provides the maximum benefit to the patient;
said discount payer providing a payment to said patient for acquisition of the pharmaceutical product; and
said patient receiving said payment, said payment received in the form of cash to the patient or as a credit to the discount manager for future use by the patient.

7. The method of claim 6 wherein said discount payer is a covered entity under Rule 304B.

8. The method of claim 7 wherein said discount payer provides a portion of a discount received under Rule 304B to the patient.

9. The method of claim 6 wherein said discount payer is a charitable organization desiring to provide payment to said patient.

10. The method of claim 6 wherein said discount payer is a private fundraiser desiring to provide payment to said patient.

11. The method of claim 6 further comprising the step of said discount manager selecting said pharmaceutical reimbursement or discount program that ensures compliance with tax laws.

12. The method of claim 11 wherein said discount payer is a covered entity under Rule 304B.

13. The method of claim 12 wherein said discount payer provides a portion of a discount received under Rule 304B to the patient.

14. The method of claim 11 wherein said discount payer is a charitable organization desiring to provide payment to said patient.

15. The method of claim 11 wherein said discount payer is a private fundraiser desiring to provide payment to said patient.

16. A system for determining eligibility of a patient for receiving a payment from a discount payer relating to the cost of a pharmaceutical product, the discount payer having a computing system including a processor, a memory, and an internet connection, the system comprising:
a discount manager having a processor, a memory, and an internet connection, said discount manager being further interconnected with each of said discount payer and discount recipient, the discount manager memory interconnected with said processor having instructions stored thereon which, when executed by said processor, causes the processor to perform operations comprising:
identifying, by a data reconciliation system of said discount manager, whether said patient has any healthcare insurance;
evaluating, by the data reconciliation system of said discount manager, whether the patient is eligible for reimbursement or credit relating to the pharmaceutical product under any pharmaceutical reimbursement or discount program;
selecting, by the data reconciliation system of said discount manager, said pharmaceutical reimbursement or discount program that provides the maximum benefit to the patient;
providing, by the data reconciliation system of said discount manager, a payment to said patient for acquisition of the pharmaceutical product.

17. A system for effectuating a payment to a patient relating to the cost of pharmaceutical product between a discount payer, having a computing system including a processor, a memory, and an internet connection, and a patient, the system comprising:
a discount manager for facilitating payment between the discount payer and the patient, the discount manager having a computing system including a processor, a memory, and an internet connection, the discount manager having a relationship with said discount payer and a relationship with said patient;
a patient-facing app provided by the discount manager, the app downloadable to and executable by an electronic device accessible to said patient, the app being interconnected with a cloud-based network having a database storage for storing an account relating to said patient and for storing any healthcare insurance information relating to said patient;
a rules matrix maintained by said discount manager and interconnected with said app, the rules matrix including benefit information relating to any healthcare insurance of said patient;
a virtual wallet residing within the app, the virtual wallet being configured to receive and hold payment or credit relating to said pharmaceutical product from said discount payer and accessible to said patient;
wherein the discount manager facilitates payment from said discount payer to said patient via said virtual wallet based on applicable benefit rules contained within said rules matrix.

18. The system of claim 17 wherein the payment from the discount payer to the patient flows directly through the discount manager.

19. The system of claim 17 wherein the rules matrix further includes tax rules to ensure compliance with tax law.

20. The system of claim 17 wherein the discount manager facilitates payment from the discount payer directly to the virtual wallet of the patient.
